(19) United States
(12) Patent Application Publication
(10) Pub. No.: US 2025/0325212 A1
(43) Pub. Date: Oct. 23, 2025

(54) ELECTRODE ATTACHMENT FOR MEDICAL EVALUATION

(71) Applicant: Worcester Polytechnic Institute, Worcester, MA (US)

(72) Inventors: Brenton D. Faber, Worcester, MA (US); Lauren B. Averka, Sutton, MA (US); Kellie Bushe, Holden, MA (US); Abigail C. Gallagher, Attleboro Falls, MA (US); Abbigail L. Poland, Tiverton, RI (US)

(21) Appl. No.: 19/184,340

(22) Filed: Apr. 21, 2025

Related U.S. Application Data
(60) Provisional application No. 63/636,288, filed on Apr. 19, 2024.

(51) Int. Cl. A61B 5/282

(52) U.S. Cl. CPC A61B 5/282; A61B 2562/04; A61B 2562/227

(57) ABSTRACT

An electrode placement device allows placement and fixation of electrocardiogram (EKG) electrodes. Proper adherence of electrodes to predetermined positions on the epidermal surface is facilitated by a plurality of elongated placement members extending from a base that align with the electrodes. Each electrode is biased against the patient sensing region, typically the chest of an EKG patient, by prongs flanking the electrode at the distal end of each of the placement members. Flanking prongs engage an outer perimeter of a flexible, skin placed electrode, while a gap between the prongs allows for a signal wire to the electrically conductive center. Problems with weak adhesion from sweat, dirt or poor adhesive are avoided by a modest biasing force imposed equally on all electrodes from manual pressure applied to the base.

### RELATED APPLICATIONS

This patent application claims the benefit under 35 U.S.C. § 119 (e) of U.S. Provisional Patent App. No. 63/636,288, filed Apr. 19, 2024, entitled “ELECTRODE ATTACHMENT FOR MEDICAL EVALUATION,” incorporated herein by reference in entirety.

### BACKGROUND

Measurement of electrical impulses can be used to assess physiological health parameters based on the minute signals transmitted in the human CNS (central nervous system). One use of electrical impulse measurement is an electrocardiogram (EKG), which is a medical diagnostic tool that is utilized to assess heart functioning by measuring the changes of electrical signals spreading through the heart as it contracts. EKGs are typically conducted by attaching small, sticky electrode patches to specific locations on the chest, arms, and legs of the patient. The electrical activity of the heart is detected by the electrodes and changes in the electrical activity are recorded by the EKG machine, which traditionally draws a trace onto a moving piece of electrocardiogramaper, however other renderings may be employed. The electrocardiogramaper has time plotted on the x-axis, voltage plotted on the y-axis, and larger and smaller squares dividing the axes into smaller increments. A properly beating heart will be coordinated by electrical impulses to different parts of the heart in order to keep blood flowing in the direction it should. Therefore, any irregularities in an EKG reading can be indicative of heart-related conditions; for instance, narrowing of coronary arteries, myocardial infarctions, or atrial fibrillation

### SUMMARY

An electrode placement device allows placement and fixation of electrocardiogram (EKG) electrodes. Proper adherence of electrodes to predetermined positions on the epidermal surface is facilitated by a plurality of elongated placement members extending from a base that align with the respective electrodes. Each electrode is biased against the patient sensing region, typically the chest of an EKG patient, by prongs flanking the electrode at the distal end of each of the placement members. Flanking prongs engage an outer perimeter of a flexible, skin placed electrode, while a gap between the prongs allows for a signal wire to the electrically conductive center. Problems with weak adhesion from sweat, dirt or poor adhesive are avoided by a modest biasing force imposed equally on all electrodes from manual pressure applied to the base.

Configurations herein are based, in part, on the observation that EKGs are a common diagnostic and evaluation medium for suspected coronary anomalies, and can be administered with portable equipment by first responders for exigent occurrences. Unfortunately, conventional approaches to EKG administration suffer from the shortcoming that adhesive electrodes, formed from a conductive patch surrounded by a flexible material, require proper fixation at a predetermined chest position for proper readings. Epidermal (skin) conditions such as excessive sweating, dirt, or adverse temperatures can affect proper adhesion. In particular, diaphoresis, which is a medical definition of excessive sweating due to an underlying health condition, episode, or medication, can be particularly problematic. When a diaphoretic patient is experiencing excessive sweating, electrodes from the EKG tend to slide from the correct position due to the excessive perspiration on the skin. When these electrodes are misplaced, the EKG is unable to accurately read the heart's electrical signals. This frequently causes misdiagnosis of cardiac problems, which can lead to negative health outcomes for patients.

Accordingly, configurations herein substantially overcome the shortcomings of conventional EKG administration by providing an electrode placement device that extends rigid placement members from a handheld base to the position of properly aligned electrodes and transfers a biasing force from the base simultaneously to all electrodes for assuring uninterrupted positioning for proper EKG reading.

In further detail, configurations herein present a therapeutic electrode placement device with a base configured for interactive manual engagement, and a plurality of elongated placement members extending from the base, such that each of the placement members has a terminal end adapted for engaging an electrode. A pair of prongs at each respective terminal end flank the electrode and are configured to bias a downward force onto the electrode and accommodate an electrical signal wire to the electrode. Each of the elongated placement members disposes the respective terminal end at a specific chest position for simultaneously biasing the corresponding electrode against a patient chest region for EKG sensing.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a medical monitoring environment suitable for use with configurations herein;

FIG. 2 shows an example configuration of the electrode placement device engaged with a patient;

FIGS. 3A and 3B shows the working principle of the electrode engagement with a patient sensing region using prongs of the electrode placement device of FIG. 2;

FIGS. 4A-4E show examples of prongs as in FIGS. 3A-3B for engaging the patient sensing region;

FIGS. 5A-5B show elongated placement members for attachment to the prongs of FIGS. 3A-4E;

FIG. 6 shows a base attached to a set of placement members for locating the prongs of the electrode placement device; and

FIG. 7 shows the electrode placement device engaged with electrodes biased on a patient sensing surface.

### DETAILED DESCRIPTION

An electrocardiogram (EKG) measures and records electrical activity of the heart, and may be performed by first responders or emergency medical services (EMS). An EKG test produces an EKG reading from which an assessment of cardiac function and a diagnosis of a heart condition can be made. Time is often a critical factor in treatment intervention of cardiac events, therefore producing an EKG reading in order to diagnose life threatening conditions as quickly as possible reduces the risk of delayed treatment, helping to prevent patient fatality.

In a typical EKG test, electrical activity in the heart is measured using electrodes and changes in electrical activity are recorded by an EKG machine, which draws a trace representing the electrical voltage signals as recorded by the electrodes. Electrodes are small, generally adhesive patches that are adhered to specific locations on a patient such as the arms, legs, and chest. A typical electrode is comprised of a round flexible backing with a conductive layer that adheres to a patient's skin (usually made of gel). A signal wire or lead is connected to the electrode at a conductive metal button, typically in the center of the backing of the electrode.

The electrodes are connected to the EKG machine by signal wires that conduct voltage from the electrodes to the EKG machine. Therefore, since an electrical voltage is being measured, it is critical that all locations along the electrical path have a low resistance. As an example, if there is poor conduction between the electrodes and the patient, a bad EKG reading will be produced by the EKG test, compromising an ability to make a proper diagnosis.

To solve this problem, a therapeutic electrode placement device may be used to bias electrodes on a patient's sensing region. Such a device may have a base configured for manual engagement, and a plurality of elongated placement members extending from the base with a terminal end of the placement members adapted for engaging an electrode. At least one prong at the respective terminal end of the placement members is configured to bias a downward force onto the electrode and accommodate an electrical signal wire to the electrode. Each of the elongated placement members therefore properly dispose the respective terminal end while simultaneously biasing the corresponding electrode against a patient sensing region, typically a chest region for a standard EKG.

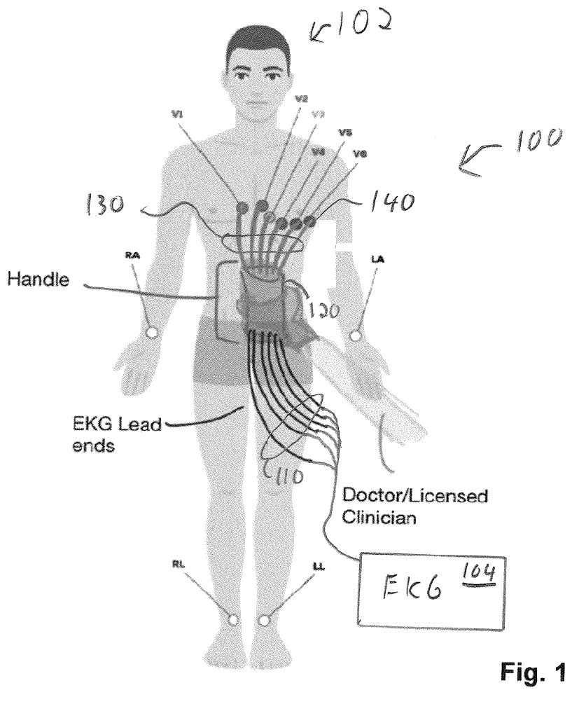

Now more specifically, in reference to the figures, FIG. 1 is a context diagram of a medical monitoring environment suitable for use with configurations herein. A therapeutic electrode placement device 100 is manually operated by an operator such as doctor or licensed clinician for use on a patient. Alternate approaches may include passive fixation by a strap or brace, or automated placement with a robotic member. During a manual engagement of the device 100, the operator (such as doctor or licensed clinician) engages a base 120 configured for interactive manual engagement of device 100 towards a patient 102 in order to conduct an EKG test.

As shown in FIG. 1, signal wires 110 from an EKG machine 104 pass through the base 120 of device 100 and into one or more elongated placement members 130, extending from the base 120. As in a typically EKG test, at the terminal end of each of the placement members 130, one or more electrodes 140 conduct electrical heart activity in the patient 102. In one example, the electrodes 140 are placed on the chest of the patient 102 close to the heart and conduct electrical signals to be recorded by the EKG machine 104. This configuration is typical of EMS responses; however, EKG readings can be taken from other locations on a patient, including, but not limited to the arms and legs. Other configurations of the device 100 can include electrodes placed in various locations on the body such as the arms and legs or some combination of such locations.

A typical EKG test relies on adhesion between electrodes and a patient's skin. However, electrode slippage frequently occurs as a result of excessive sweating (diaphoresis) of the patient, which causes the electrodes to not adhere to the patient's skin. Current products used by medical professionals to attempt to create a higher adhesion to between electrodes and diaphoretic skin use a “stickier” adhesive or use more adhesive gel; however, such products are more expensive and while they attempt to provide more adhesion, conventional approaches find that they do not provide enough adhesion to produce an accurate EKG reading.

During the manual operation of device 100, the operator (doctor/licensed clinician or other suitable operator such as EMS responder) holds the device 100 and applies a modest force on the base 120 or handle towards the patient's chest. The force supplied by the operator is passed through the placement members 130 to the electrodes 140 and produces a pressure between the electrodes 140 and the patient's chest. The pressure between the electrodes 140 and the patient ensures that there is a high electrical conductivity between the electrodes 140 and the patient's chest, which is beneficial to produce an accurate EKG reading during the EKG test.

The electrodes 140 used in device 100 as shown in FIG. 1 are typical EKG electrodes and rely on the pressure transferred from the operator through the device 100 to the pads of electrodes 140 for proper electrical contact instead of using a high adhesive gel. While diaphoretic electrodes may be available, the disclosed approach mitigates the need for their availability and use. Rather, a manually created pressure prevents slippage of the electrodes from the skin to reliably obtain a proper EKG reading during an EKG test.

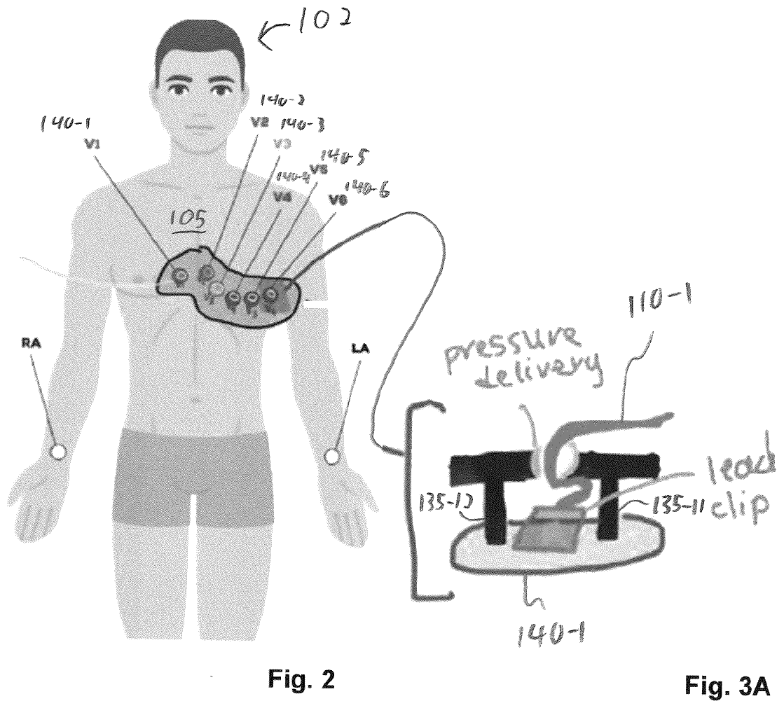

FIG. 2 shows an example configuration of the electrode placement device engaged with a patient. Referring to FIGS. 1 and 2, in the example of FIG. 2, 6 electrodes 140-1 . . . 140-6 (140 generally) are arranged on the patient's chest in locations labeled respectively V1-V6. The electrodes are arranged in a configuration typical for an EKG test to measure the heart's electrical signals; however, in other configurations, electrodes may be placed in other locations on the body such as the arms and legs. Additionally, while 6 electrodes are used in this example configuration, in other configurations, any number of electrodes may be used.

Each of the electrodes 140, using electrode 140-1 as an example. corresponds to the EKG test location V1, and the device 100 is arranged to place the electrode 140-1, which is bound or engaged to placement member 130-1, on the test location V1. Similarly, electrode 140-2 corresponds to the EKG test location V2, and the device 100 is arranged to place the electrode 140-2, which is bound to placement member 130-2, on the test location V2. Similarly, electrodes 140-3 . . . 140-6 are arranged at test locations V3. . . . V6 respectively by placement members 130-3 . . . 130-6 respectively.

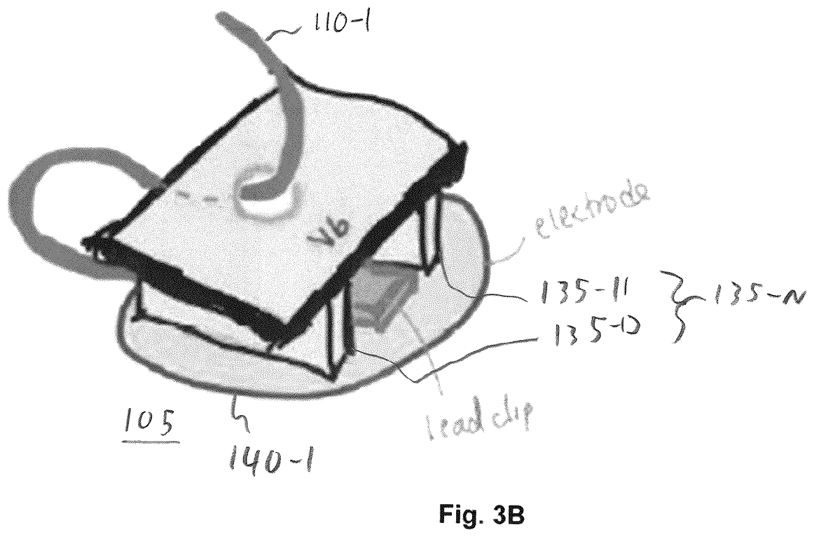

FIGS. 3A and 3B shows the working principle of the electrode engagement with a patient sensing region using prongs of the electrode placement device of FIG. 2. In the example in FIG. 3A, an illustration shows a particular engagement of an electrode 140-1 engagement with a placement member extending from the base. Pictured is the terminal end of the placement member, which is comprised of a plurality of prongs 135-11 . . . 135-12 (135 generally). The prongs 135 are engaged with the electrode 140-1. Additionally, a signal wire 110-1 to the electrode 140-1 is flanked by the prongs 135.

During operation, pressure is delivered from the placement member through the top of the prongs 135 and to the electrode 140-1. When placed on the patient's chest (or another suitable location) as shown in FIG. 2, increased contact between the electrode and the patient's skin produces a higher electrical conductivity between the patient skin and the electrode 140-1, allowing a more desirable signal (with less noise and higher voltage) to pass through the signal wire 110-1 to the EKG machine 104.

As shown in FIGS. 3A and 3B, a plurality of prongs may be used to create a consistent pressure between the electrode 140-1 and the patient's skin. The plurality of prongs at each terminal end of a placement member are configured to flank the electrode in a biased position against the patient sensing region. In the illustrated example, there are 2 prongs 135-11 and 135-12 (collectively 135-N), one on either side of the metal part of the electrode 140-1, which is connected to the signal wire 110-1. In other examples, one prong, three prongs, or any number of prongs may be used.

Referring specifically to the single electrode 140 of FIG. 3B, the 2 prongs 135-11 and 135-12 form a pair of opposed prongs 135-N at the terminal end of each placement member 130, with the prongs 135 flanking the signal wire 110-1 which is connected to the electrode 140-1. During operation, this configuration imposes a biasing face on the electrode 140-1 from the pair of opposed prongs 135-N on opposing sides of the signal wire 110-1.

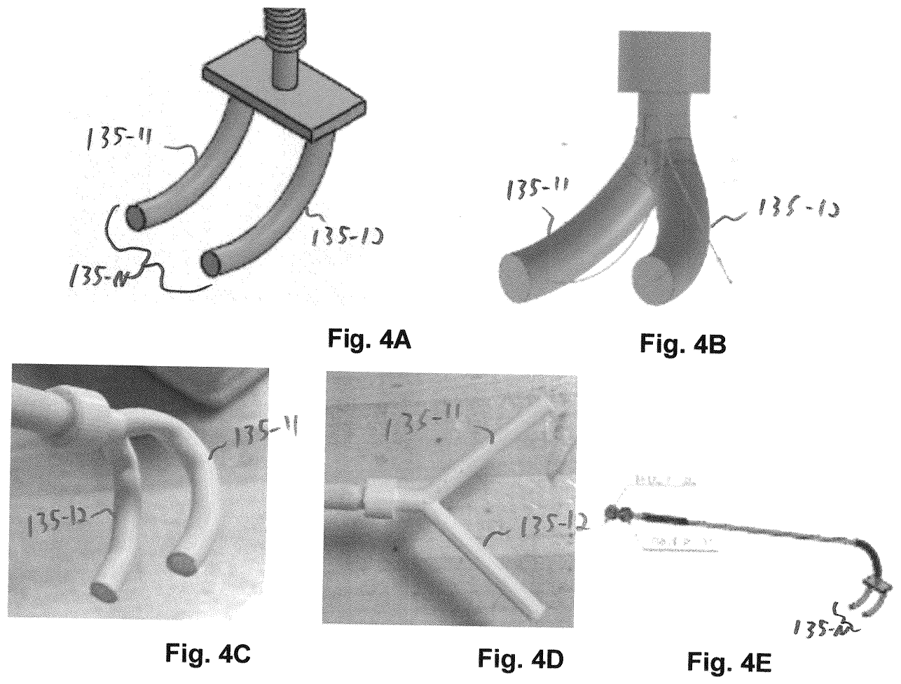

FIGS. 4A-4E show examples of prongs as in FIGS. 3A-3B for engaging the patient sensing region. The example prongs 135-N in FIG. 4A is a 3D rendering of an example set of prongs 135 that may be used in the device 100 to exert pressure from a corresponding placement member to a corresponding electrode 140. In this example, 2 prongs 135-11 and 135-12 extend from the end of the placement member 130 in parallel. Both prongs extend outward from the end of the placement member and form an inverted bend in parallel with one another, appearing as 2 identical hook shapes. This leaves room in between prongs 135-11 and 135-12 for a signal wire to connect to an electrode. The electrode is to be placed tangentially to the prongs 135-11 and 135-12 on the exterior of the inverted bend.

While the pair of opposed prongs 135-N initially extend from the end of a placement member 130 toward the patient's sensing surface, as a result of the inverted bend in the prongs 135-1, the pair of opposed prongs 135-1 eventually run substantially perpendicular to the patient sensing surface. With the signal wire 110 fed between the opposed prongs 135-11, 135-12, the surfaces of the prongs 135, where the inverted bend is running substantially perpendicular to the patient sensing surface 105, flank the corresponding electrode 140, and exert a biasing force on the electrode towards the patient sensing surface 105.

FIG. 4B is another example 3D rendering of prongs 135-1 that may be used in device 100 similarly to FIG. 4A. In this example, 2 prongs 135-11 and 135-12 extend from the end of a placement member. Unlike in the example in FIG. 4A, at the end of the placement member the prongs first extend outward as one cylinder and then split into the 2 prongs 135-11 and 135-12. In this configuration, the prongs form a bifurcated extension from the terminal end of each respective placement member.

This example reduces the complexity of the prong assembly (typically a pair) by lowering the number of individual manufactured parts when compared to the prongs in FIG. 4A. In this example, there are interior threads on the top of opposed prongs 135-1. The prongs 135-1 may be produced as one piece and then bound to the terminal end of the placement member by screwing together interior threads on the prongs 135-N to exterior threads on the terminal end of the placement member. While this is one configuration for binding together the prongs 135-N and the placement member, other binding methods such as gluing, taping, welding, etc., may be used.

FIGS. 4C and 4D are examples of manufactured prongs 135-N to be used in device 100.

The example in FIG. 4D shows an intermediate step in the manufacturing process. In this example, the pair of opposed prongs 135-1 are comprised of PLA (Polylactic Acid), a common printing filament, and 3D printed using an appropriate printer. The 3D printer prints straight Y shaped pipes with an interior threaded top to connect a placement member. This example uses PLA produced by a 3D printer, although any suitable material and manufacturing process may be used to produce the prongs. The 2 prongs 135-11 and 135-12 extend from the terminal end of the placement member and form a bifurcated extension as in the example rendering in FIG. 4B. However, unlike in the example in FIG. 4B, the prongs are not curved. FIG. 4C shows the same prongs 135 as in FIG. 4D after a curving step in the manufacturing process occurs. This may be done by heating the prongs 135 with a heat gun and manually shaping the prongs into a desired shape as shown in FIG. 4C. In other examples, another suitable manual, or automatic, manufacturing process may be used to shape the prongs such as bending or starting with a curved stock material. The final prongs as shown in FIG. 4C are screwed by the interior threads to exterior threads on the terminal end of the placement member and are used by device 100 to exert force from the placement member to a flanked electrode.

FIG. 4E is a 3D rendering of an example configuration of how a set of prongs could attach to a placement member. In this example, a placement member 130-1, which may include one or more articulations in order to position an electrode on a patient sensing region, has prongs 135-1 attached to it. In this case, the prongs 135-1 are as described in FIG. 4A and extend from the end of the placement member 130-1. The prongs 135-1 may be screwed, glued, taped, or otherwise fastened in some suitable manner to the placement member 130-1, or the prongs 135-1 and placement member 130-1 may be one conjoined piece. In the example in FIG. 4E the prongs 135-1 has a threaded end that may be screwed onto the placement member 130-1.

With the prongs positioned over a patient's sensing region 105, the plurality of prongs 135 such as shown in FIGS. 4A-4E are configured to flank an electrode 140 against the patient's sensing region. In one example, as illustrated in FIGS. 4A-4E, the plurality of prongs 135 comprises a pair of opposed prongs 135-N at each terminal end of the placement member 130-1, with the prongs 135 flanking the signal wire 110 connected to the and impose a biasing force on the on opposed sides of the electrode 140. As an operator exerts a modest downward force on the device, the electrode bound to the prongs is biased against the patient's skin, creating increased contact between the electrode and the patient's epidermis.

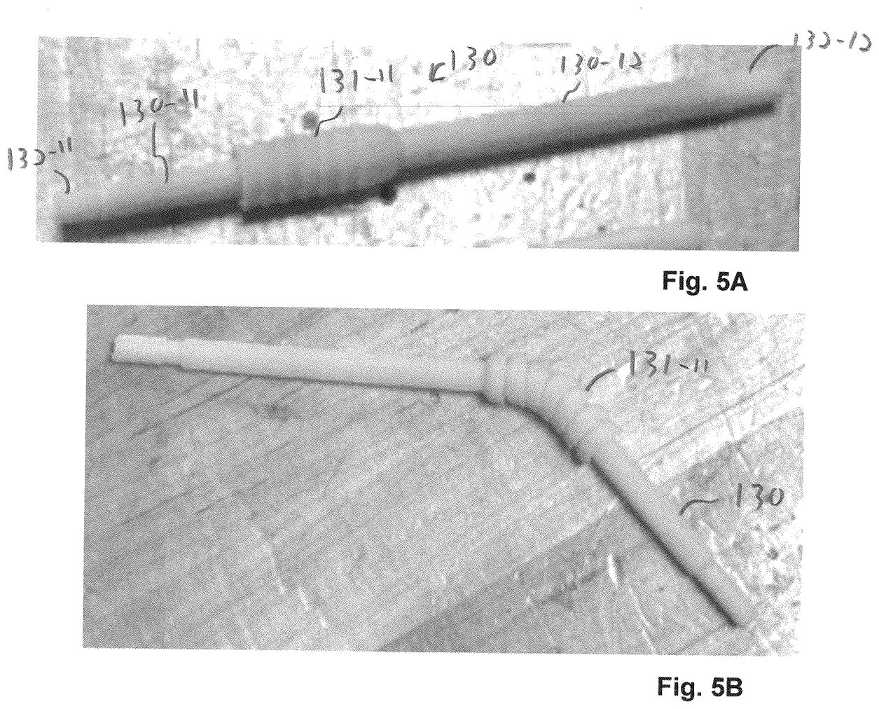

FIGS. 5A-5B show elongated placement members for attachment to the prongs of FIGS. 3A-4E. In this example, a placement member 130-1 is composed of ABS and manufactured using a 3D printer. ABS is chosen due to its rigidity and ability to bend under high temperatures. In other configurations, the placement member may be composed of PLA, a flexible material, or another suitable material.

Due to space limitations of typical 3D printers, one placement member 130-1 may be 3D printed in multiple parts. FIG. 5 shows placement member section 130-11 which, which when combined with zero or more other placement member sections can be assembled to form placement member 130-1. As shown in FIGS. 5A-5B, placement member section 130-11 may include one or more threaded ends, 132-11 and 132-12, which are used to connect placement member sections 130-11, 130-12 etc., during assembly to form placement member 130. Threaded ends 132-11 and 132-12 may be inner threads, outer threads, or a combination of both.

Additionally in this example, placement member section 130-11 is comprised of hollow tubing of ABS as manufactured by a 3D printer. The hollow tubing allows for a signal wire (not pictured) to pass through the one or more sections 130-11, 130-12, etc., of the placement member 130-1. In this example, the tubing has a thickness of 2.0 mm which is chosen to withstand forces passed through the placement member section 130-11 during the operation of the device.

Further in this example, placement member section 130-11 may include 1 or more articulations 131-11, 131-12 (131 generally). As in FIGS. 5A-5B, articulation 131-11 is used to create curvature in the placement member section 130-11. The articulation 131-11 is manufactured by creating a series of segments of varying diameter, at least as large as the diameter of the placement member 130-1, in the 3D printed ABS, which allows it to contract and expand to the desired shape of the articulation 131-11, similar to a plastic straw. A 3D printer produces the straight placement member section 130-11 with articulation 131-11 as in FIG. 5A. Subsequently, a heat gun heats the ABS of the articulation 131-11 to its deflection temperature range (80° C.-100° C.). The articulation 131-11 is curved into a desired shape as the example in FIG. 5B of the articulation 131-11 depicts. When the articulation 131-11 cools down below its deflection temperature range, it resumes its rigidity necessary for use during operation of the device.

The articulations 131 are used to dispose and locate the terminal ends of the placement members 130 to the patient sensing region 105 based on a common distance from the base 120 of the device 100. As in the example in FIG. 2, one configuration may have six locations V1-V6 on the patient's chest as the desired electrode location. Since there is one common location for the base 120, the electrodes must be disposed by the placement members 130 and their respective articulations 131 to their relative location to the base 120. Thus, zero or more articulations dispose the terminal ends and therefore the electrodes 140 based on this common distance to the patient sensing region.

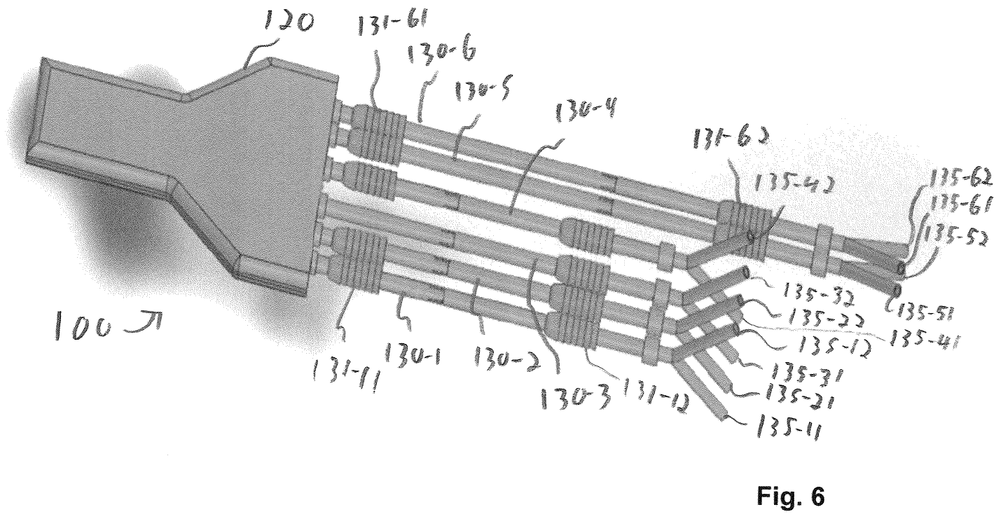

FIG. 6 shows a base attached to a set of placement members for locating the prongs of the electrode placement device. In this example, a 3D rendering of the device 100 includes a base 120, placement members 130 including placement members 130-1, 130-2, etc. and corresponding prongs 135-1, 135-2, etc.

Each placement member, such as placement member 130-1 is connected to the base via a suitable method such as screwing between exterior threads on the placement member 130-1 and interior threads on the base 120. During operation of the electrode placement device, an operator biases a downward force on the base 120, which passes from base 120 to the placement members 130, in order to bias a corresponding electrode 140 at the terminal end of each placement member 130 towards a patient sensing region. Therefore, a rigid attachment from the base 120 to each of the respective placement members 130, is preferable for the base 120 to transfer the biasing force to placement members 130. The base 120 may also include a threaded receptacle for receiving each placement member 130.

Further in this example, each placement member 130-1, 130-2, etc., may be a different length in order to accurately position the prong 135 to the desired sensing region on the patient. For example, in this configuration, placement members 130-1, 131-2, and 130-4 are all a first length, placement member 130-3 is a second length, and placement members 130-5, 130-6 are a third length.

Still further in this example, each placement member 130 may have a different number of articulations in order to accurately position the prongs of the electrode placement device with respect to the desired patient sensing region. Placement members 130-1, 130-2, and 130-4 . . . 130-6 each have 2 articulations while placement member 130-3 has 1 articulation. In other configurations, one or more placement members may have zero or any number of articulations necessary to position the prongs with respect to the patient sensing region.

The configuration of varying lengths of placement members 130, along with varying numbers of articulations 131, is such that an aggregation of each of the articulations 131 of the placement members 130, extending from the base 120, disposes the terminal end in alignment with the patient sensing surface. As an operator applies a downwards force on the base 120, the placement members 130 bias a substantially equal force from the base 120 towards the patient sensing surface.

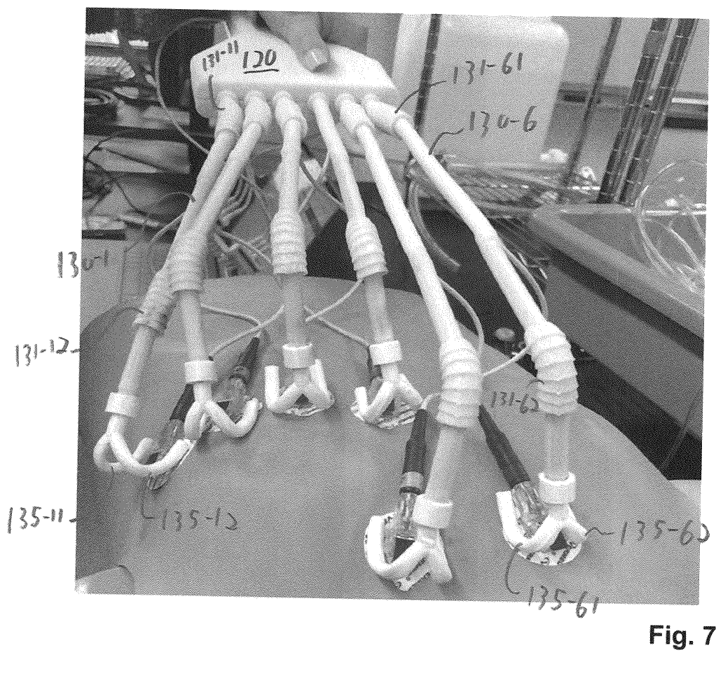

FIG. 7 shows the electrode placement device engaged with electrodes biased on a patient sensing surface. In this example, a fully manufactured prototype of an electrode placement device 100 is being used on a patient, as an example of device 100 operation. An operator holds the base 120 of the device 100 over the patient to perform an EKG test on the patient. Six placement members 130 extend from the base 120, including members 130-1 . . . 130-6. The six placement members 130 each have a pair of opposed prongs 135-N which act to hold the respective six electrodes 140. Additionally, six respective signal wires 110 extend from the electrodes to the EKG machine 104. In this configuration, the signal wires 110 are exterior to the placement members 130; however, in other configurations, the signal wires 110 may be passed through the interior of each of the respective placement members 130. This may be done for wire organization, to protect the signal wires 130 and to ensure the signal wires do not become entangled with any obstacles during operation of the device 100.

To perform an EKG test on the patient, the operator exerts a modest downward force on the base 120 which is configured for interactive manual engagement. This downward force is passed through the plurality of placement members 130 extending from the base 120, and in turn to the terminal end of the placement members 130 and prongs 135 which are adapted for engaging the electrodes 140. A pair of prongs 135 at each respective terminal end of the placement members 130 are configured to bias the downward force exerted by the operator onto the electrodes 140 and accommodate an electrical signal wire 110 to the electrodes 130. As the downward force is applied, each of the elongated placement members 130 simultaneously bias the corresponding electrode 140 against the patient's chest (or other patient sensing region), via the respective terminal end of each placement member 130.

As the downwards force biases the electrodes 130 against the patient's chest, additional surface area between each electrode 130 and the patient's skin leads to a greater conductivity of electrical signals passed from the patient to the attached signal wires 110. With a greater conductivity, a better EKG reading can be produced by an EKG machine as the electrical signals that reach the EKG machine more accurately match the electrical signals in the patient, specifically heart activity signals. For patients with diaphoresis, EMS often have difficulty obtaining an accurate EKG reading due to poor adhesion between the electrodes and the patient's skin. This is especially important for EMS (or other professionals) to diagnose heart conditions that may threaten the patient's health, reducing the time necessary to administer potentially lifesaving treatment to the patient.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A therapeutic electrode placement device, comprising:
a base configured for interactive manual engagement;
a plurality of elongated placement members extending from the base, each of the placement members having a terminal end adapted for engaging an electrode; and
at least one prong at each respective terminal end, the prong configured to bias a downward force onto the electrode and accommodate an electrical signal wire to the electrode;
each of the elongated placement members disposing the respective terminal end simultaneously biasing the corresponding electrode against a patient sensing region.

2. The device of claim 1 further comprising a plurality of prongs at each terminal end, the plurality of prongs configured to flank the electrode in a biased position against the patient sensing region.

3. The device of claim 1 wherein each of the placement members has zero or more articulations, the articulations disposing the terminal ends based on a common distance to the patient sensing region.

4. The device of claim 3 wherein the articulations include a series of segments of varying diameter at least as large as the diameter of the placement member.

5. The device of claim 1 further comprising a rigid attachment from the base to each of the respective placement members, the base configured to transfer the biasing force to the placement members.

6. The device of claim 2 wherein the plurality of prongs further comprise a pair of opposed prongs at each terminal end, the prongs flanking a signal wire to the electrode and imposing the biasing force on the electrode on opposed sides of the signal wire.

7. The device of claim 6 wherein the pair of opposed prongs extend from the terminal end for extending towards the patient sensing region and forming an inverted bend to a run substantially perpendicular to the patient sensing surface.

8. The device of claim 2 wherein the plurality of prongs form a bifurcated extension from the respective terminal end.

9. The device of claim 4 wherein an aggregation of each of the articulations of the placement members extending from the base disposes the terminal end in alignment with the patient sensing surface for biasing a substantially equal force from the base towards the patient sensing surface.

10. A method for administering an electrode based diagnostic process, comprising:
extending a plurality of elongated placement members extending from a base, each of the placement members having a terminal end adapted for engaging an electrode, the base configured for interactive manual engagement;
attaching at least one prong at each respective terminal end, the prong configured to bias a downward force onto the electrode and accommodate an electrical signal wire to the electrode; and
applying a force to the base such that each of the elongated placement members disposes the respective terminal end simultaneously biasing the corresponding electrode against a patient sensing region.
